(19) United States
(12) Patent Application Publication
(10) Pub. No.: US 2012/0294834 A1
(43) Pub. Date: Nov. 22, 2012

(54) REGENERATION OF, REESTABLISHING FUNCTION IN AND REPLACING MICROVASCULATURE IN ORGANS AND TISSUES

(75) Inventor: Leon G. Fine, Los Angeles, CA (US)

(73) Assignee: CEDARS-SINAI MEDICAL CENTER, Los Angeles, CA (US)

(21) Appl. No.: 13/519,482

(22) PCT Filed: Dec. 30, 2010
(86) PCT No.: PCT/US10/62558

§ 371 Date: Jun. 27, 2012

Related U.S. Application Data
(60) Provisional application No. 61/291,780, filed on Dec. 31, 2009.

(51) Int. Cl. A61K 35/12; A61P 11/00; A61K 35/28; A61P 13/12; A61P 27/02; A61K 35/14; A61P 9/00; A61P 1/16

(52) U.S. Cl. 424/93.7

(57) ABSTRACT

The invention provides compositions and methods involving the use of endothelial progenitor cells (EPC) to re-generate and/or reestablish a functioning microvasculature in damaged or ischemic organs and tissue. The invention also provides compositions and methods using EPC for replacing lost function in organ and tissue that is damaged, ischemic, or scarred. Such compositions and methods may find utility in, for instance, organs or tissues that have been damaged due to lack of vasculature.

### CROSS-REFERENCE TO RELATED APPLICATIONS

The present application claims the benefit of the filing date of U.S. Provisional Application No. 61/291,780 filed Dec. 31, 2009, the disclosure of which is incorporated herein by reference in its entirety.

### STATEMENT REGARDING FEDERALLY-SPONSORED RESEARCH

This invention was made with government support under Telemedicine & Advanced Technology Research Center (TATRC) Grant No. W81XWH-09-1-0644 from the U.S. Army Medical Research and Material Command (USAMRMC). The Government has certain rights in the invention.

### FIELD OF THE INVENTION

The invention provides methods of using endothelial progenitor cells (EPC) for the regeneration of tissue and organ damage, ischemia, and scarring. Such methods find utility in the re-establishment and repair of the microvasculature of tissues and organs.

### BACKGROUND OF THE INVENTION

The scientific community is intensively focused upon a panoply of possibilities for the use of progenitor and stem cells in promoting repair in a variety of tissues and organs. Stem cells and/or progenitor cells can be isolated from many locations in the adult body, including but not limited to bone marrow, placenta, adipose tissue, lung, blood, and teeth, and can be treated in vitro to become endothelial progenitor cells (16).

For many years it has been known that a population of stem cells exists in the normal adult circulation and bone marrow. Different sub-populations of these cells can differentiate along hematopoietic lineage positive (Lin⁺) or non-hematopoietic, lineage negative (Lin−) lineages. Furthermore, the lineage negative hematopoietic stem cell (HSC) population has recently been shown to contain EPC capable of forming blood vessels in vitro and in vivo (16). These cells can participate in normal and pathological postnatal angiogenesis (17-19).

Current approaches to organ regeneration using the cells focus upon methods of achieving differentiated phenotypes from these cells, which match the parenchymal cells of the organ or tissue to be regenerated. Thus, regeneration would be the result of replacement. Since many organs and tissues have complex structures and designs (e.g. kidney nephrons), supplying stem cells that can develop into the different cell types and provide the proper structure is quite complex. Thus, there is a need in the art for tissue regeneration using stem cells that are not required to differentiate into particular cells of individual organs, but rather can repair and/or re-establish the microvascular of an organ and tissue and thereby improve perfusion and oxygenation of the damaged organ, thus allowing the existing architecture and scaffolding to be repaired.

### SUMMARY OF THE INVENTION

In an embodiment, the invention includes a method of restoring function to a chronically injured tissue or organ in a subject, comprising: providing a quantity of endothelial progenitor cells (EPC); and administering the quantity of EPC to the subject in an amount effective to restore the function and/or architecture of the chronically injured tissue or organ. The quantity of EPC may be administered to the subject by adoptive transfer. The quantity of EPC may be administered to the subject systemically or by direct administration to the tissue or organ. The tissue or organ may be a lung, a kidney, a liver, a heart, connective tissue, an eye, or a combination thereof. The subject may be a mammal. The subject may be a human. The architecture may comprise the microvasculature of the tissue or organ. The EPC may be endothelial precursor cells, endothelial cell precursors, hematopoietic stem cells, mesenchymal stem cells, embryonic stem cell lines, erythropoietic stem cells, young bone marrow cells or young cardiac microvascular endothelial cells

In another embodiment, the invention includes a method of regenerating a chronically injured tissue or organ in a subject, comprising: providing a quantity of endothelial progenitor cells (EPC); and administering the quantity of EPC to the subject in an amount effective to regenerate the chronically injured tissue or organ. The quantity of EPC may be administered to the subject by adoptive transfer. The quantity of EPC may be administered to the subject systemically or by direct administration to the tissue or organ. The tissue or organ may be a lung, a kidney, a liver, a heart, connective tissue, an eye, or a combination thereof. The architecture may comprise the microvasculature of the tissue or organ. The subject may be a mammal The subject may be a human. The EPC may be endothelial precursor cells, endothelial cell precursors, hematopoietic stem cells, mesenchymal stem cells, embryonic stem cell lines, erythropoietic stem cells, young bone marrow cells or young cardiac microvascular endothelial cells

In another embodiment, the invention includes a composition for restoring function to a chronically injured tissue or organ, comprising: a quantity of endothelial progenitor cells (EPC) in an amount effective to restore function to the tissue or organ; and a pharmaceutically acceptable carrier. In another embodiment, the invention includes a composition for regenerating a chronically injured tissue or organ, comprising: a quantity of endothelial progenitor cells (EPC) in an amount effective to regenerate the tissue or organ; and a pharmaceutically acceptable carrier.

In another embodiment, the invention includes a composition for the regeneration of the microvasculature of a chronically injured tissue or organ, comprising: a quantity of endothelial progenitor cells (EPC) in an amount effective to regenerate the microvasculature of the tissue or organ; and a pharmaceutically acceptable carrier.

### DETAILED DESCRIPTION OF THE INVENTION

All references cited herein are incorporated by reference in their entirety as though fully set forth. Unless defined otherwise, technical and scientific terms used herein have the same meaning as commonly understood by one of ordinary skill in the art to which this invention belongs. Singleton et al., Dictionary of Microbiology and Molecular Biology 3^{rd} ed., J. Wiley & Sons (New York, N.Y. 2001); March, Advanced Organic Chemistry Reactions, Mechanisms and Structure 5^{th} ed., J. Wiley & Sons (New York, N.Y. 2001); and Sambrook and Russel, Molecular Cloning: A Laboratory Manual 3rd ed., Cold Spring Harbor Laboratory Press (Cold Spring Harbor, N.Y. 2001), provide one skilled in the art with a general guide to many of the terms used in the present application.

One skilled in the art will recognize many methods and materials similar or equivalent to those described herein, which could be used in the practice of the present invention. The present invention is in no way limited to the methods and materials described. For purposes of the present invention, the following terms are defined below.

The term “endothelial progenitor cell” (EPC) refers to any cell that can differentiate into endothelial cells; the cells that make up the lining of blood vessels. EPC promote neogenesis or angiogenesis of vascular tissues and can be used to revascularize tissues and organs. Examples of EPC include, but are not limited to, endothelial precursor cells, endothelial cell precursors, hematopoietic stem cells (HSC), mesenchymal stem cells (MSC) embryonic stem cell lines, erythropoietic stem cells, young bone marrow cells, young cardiac microvascular endothelial cells and other types of stem cells. The term “stem cells,” as used herein, are cells which are not terminally differentiated and are therefore able to produce cells of other types. Stem cells are divided into three types, including totipotent, pluripotent, and multipotent. “Totipotent stem cells” can grow and differentiate into any cell in the body and thus, can form the cells and tissues of an entire organism. “Pluripotent stem cells” are capable of self-renewal and differentiation into more than one cell or tissue type. “Multipotent stem cells” are clonal cells that are capable of self-renewal, as well as differentiation into adult cell or tissue types. Multipotent stem cell differentiation may involve an intermediate stage of differentiation into progenitor cells or blast cells of reduced differentiation potential, but are still capable of maturing into different cells of a specific lineage. The term “stem cells,” as used herein, refers to pluripotent stem cells and multipotent stem cells capable of self-renewal and differentiation. “Adult stem cells” are a population of stem cells found in adult organisms with some potential for self-renewal and are capable of differentiation into multiple cell types. “Hematopoiesis” refers to the process of blood cell development and homeostasis. Prenatally, hematopoiesis occurs in the yolk sack, then liver, and eventually the bone marrow. In normal adults, it occurs primarily in bone marrow and lymphatic tissues. All blood cells develop from pluripotent stem cells, which are committed to three, two, or one hematopoietic differentiation pathways. The term “hematopoietic stem cells,” as used herein, means multipotent stem cells that are capable of eventually differentiating into all blood cells including, erythrocytes, leukocytes, megakaryocytes, and platelets. This may involve an intermediate stage of differentiation into progenitor cells or blast cells. The term “hematopoietic progenitors,” “progenitor cells” or “blast cells” are used interchangeably in the present invention and describe maturing HSCs with reduced differentiation potential, but are still capable of maturing into different cells of a specific lineage, such as myeloid or lymphoid lineage. “Hematopoietic progenitors” include erythroid burst forming units, granulocyte, erythroid, macrophage, megakaryocyte colony forming units, granulocyte, erythroid, macrophage, and granulocyte macrophage colony-forming units.

“Mammal” as used herein refers to any member of the class Mammalia, including, without limitation, humans and nonhuman primates such as chimpanzees and other apes and monkey species; farm animals such as cattle, sheep, pigs, goats and horses; domestic mammals such as dogs and cats; laboratory animals including rodents such as mice, rats and guinea pigs, and the like. The term does not denote a particular age or sex. Thus, adult and newborn subjects, as well as fetuses, whether male or female, are intended to be included within the scope of this term.

“Chronic injury” or “chronic disease” refers to any injury or disease to tissues or organs that causes the tissue or organ to lose its function and also causes deterioration of the microvasculature in parallel with dysfunction of endothelial precursors. A chronically injured or diseased tissue or organ can be further characterized by hypoxia, ischemia, scarring, fibrosis, and/or loss of architecture.

“Regeneration” refers to the partial or entire re-establishment, restoration, or replacement of a functioning microvasculature of “chronically injured” tissues or organs. “Regenerated” tissues and organs either wholly or partially regain their original function and architecture. Regeneration can be achieved using adoptive transfer of EPC. Tissues and organs that can be regenerated include, but are not limited to kidney, liver, lung, heart, connective tissue, and eyes.

“Adoptive transfer” as used herein, refers to the process of transferring EPC to a chronically injured or diseased tissue or organ for the regeneration of the tissue or organ. EPC can be transferred systemically or by direct transfer into the tissue or organ.

“Treatment” and “treating,” as used herein refer to the regeneration of chronically injured tissues and organs. Tissues and organs have been “treated” when the original function and architecture have been restored.

“Therapeutically effective amount” as used herein refers to that amount which is capable of achieving beneficial results in a patient with chronic injury or chronic disease of a lung, a kidney, a liver, a heart, connective tissue, an eye, or a combination thereof. A therapeutically effective amount can be determined on an individual basis and will be based, at least in part, on consideration of the physiological characteristics of the mammal, the type of delivery system or therapeutic technique used and the time of administration relative to the progression of the disease.

“Pharmaceutically acceptable carriers” as used herein refer to conventional pharmaceutically acceptable carriers useful in this invention. Remington's Pharmaceutical Sciences, by E. W. Martin, Mack Publishing Co., Easton, Pa., 15th Edition (1975), describes compositions and formulations suitable for pharmaceutical delivery of the inventive compositions described herein.

In various embodiments, EPC may be provided as pharmaceutical compositions including a pharmaceutically acceptable excipient along with a therapeutically effective amount of the EPC. “Pharmaceutically acceptable excipient” means an excipient that is useful in preparing a pharmaceutical composition that is generally safe, non-toxic, and desirable, and includes excipients that are acceptable for veterinary use as well as for human pharmaceutical use. Such excipients may be solid, liquid, or semisolid.

In various embodiments, the pharmaceutical compositions according to the invention may be formulated for delivery via various routes of administration. “Route of administration” may refer to any administration pathway known in the art, including but not limited to transmucosal or parenteral. “Parenteral” refers to a route of administration that is generally associated with injection, including intraorbital, infusion, intraarterial, intracapsular, intracardiac, intradermal, intramuscular, intraperitoneal, intrapulmonary, intraspinal, intrasternal, intrathecal, intrauterine, intravenous, subarachnoid, subcapsular, subcutaneous, transmucosal, or transtracheal. Via the parenteral route, the compositions may be in the form of solutions or suspensions for infusion or for injection, or as lyophilized powders.

All examples of chronic solid organ fibrosis with loss of function show progressive loss of the interstitial microvasculature. In the kidney, the initial insult to the organ usually leads to local areas of hypoxia. Hypoxia, per se, is a profibrogenic stimulus which leads to scar formation. Scars, in turn, obliterate microvessels, leading to a downward spiral of decreased function. Substantial evidence has accrued to show that the “Chronic Hypoxia Hypothesis” has been substantiated for chronic renal diseases (9).

Attempts to regenerate chronically-diseased solid organs, by the transfer of progenitor cells which differentiate into the parenchymal phenotypes of the organ, are likely to fail in the long-term, since these cells will not survive the hypoxic environment. Resident progenitor cells suffer the same fate. Furthermore, even if transferred parenchymal cells were to survive, they would be unlikely to be able to recreate the complicated internal architecture of an organ (e.g. new nephrons, each containing up to 30 different phenotypes, cannot be formed in the adult kidney).

The Unifying Vasculogenic Hypothesis for Solid Organ Regeneration states: “Regeneration of solid organs after chronic injury and scarring can be achieved solely by restoring the microvasculature. This can be achieved by adoptively transferring endothelial progenitor cells into the organ. The ensuing improvement in microvasculature, with relief of hypoxia, will stimulate resident progenitor cells to reconstitute the vascular network of the organ. Resident parenchymal progenitor cells and differentiated cells will consequently be restored to function, which will regenerate the parenchyma of the organ, including partial remodelling of scarred areas, within its existing architectural structure.”

The inventor believes that in order to restore solid organ function after chronic injury all that is required to initiate the process is a restored microvascular circulation. There should be no need to generate and transfer stem/progenitor cells of the specialized phenotype of each organ. This would apply to organs such as kidney, liver, lungs and heart.

The present invention provides, in one embodiment, methods of using endothelial progenitor cells (EPC) to re-establish a functioning microvasculature in damaged or ischemic organs and tissue. Inventive methods and uses include, but are not limited to, treating damaged tissue with EPC that can repair the microvasculature and thus rejuvenate the existing architecture of the tissue and/or organ.

The inventor herein discloses an approach to organ regeneration which does not require differentiated parenchymal cells to replace function, but which utilizes only angiogenic progenitor cells (e.g. endothelial progenitors) to regenerate an interstitial capillary network. According to certain embodiments, this alone will restore oxygenation to the damaged tissue and allow resident cells of all phenotypes to regenerate themselves on existing scaffolds in any organ or tissue. Regeneration is thus entirely driven by restoration of microvascular integrity without the need for generation of differentiated cells of different phenotypes from stem and progenitor cells.

### Adriamycin-Associated Nephropathy (AAN)—a Kidney Damage Murine Model

Adriamycin-associated nephropathy (AAN) is a model currently used to examine the progression of kidney damage and the treatments and therapeutics that can be utilized to counteract this damage. Adriamycin is an anthracycline antibiotic that has many effects on bodily functions including depression of the bone marrow and development of cardiomyopathy and nephropathy (13-15). In particular, the nephropathy effects (AAN) contribute significantly to its toxicologic profile.

Studies have reported that murine hematopoietic stem cells demonstrate low fluorescence after staining with the dye Hoechst 33342, a characteristic which has been useful in the purification of such cells. When used in combination with other markers, such low-dye-expressing cells have been described as “side population” cells, based upon their ability to export intracellular dye rapidly (5-7). Putative stem cells from a variety of tissues appear to conform to this phenotype (8).

Many groups have further characterized this “kidney side population” of cells and demonstrated that injection of such cells is able to reduce proteinuria in a mouse model of adriamycin-nephropathy (3-5, 8).

Interestingly, a recent study has shown that the kidney-resident side population cells, capable of multilineage differentiation, as well as the main population cells (devoid of side-population cells) adoptively transferred to mice with AAN resulted in the reduction of proteinuria (2). In this study, a resident population of cells of nonhematopoietic immunotype was identified with a proximal tubular location and with the ability to differentiate into multiple lineages (2). Introduction of such cells into mice with adriamycin-nephropathy, improved glomerular filtration rates and decreased albuminuria but did not appear to integrate into renal tubules, leaving the mechanism for functional improvement unclear.

A more recent study, using the AAN model, examined whether the impairment of endothelial progenitor cell function is the basis for impaired regeneration and, if so, whether repair by exogenous endothelial precursor cells might be possible (1). Yasuda, et al. analyzed hematopoietic stem cells (HSC) and endothelial progenitor (EPC) cells derived from the kidneys of mice with AAN and reported that both resident populations were sparse (<0.1% of total cell number) in control kidneys and did not change numerically after adriamycin-induced injury. These cells demonstrated decreased viability, increased senescence and increased apoptosis, which were not attributable to loss of stromal cell-derived factor-1 (SDF-1) production, a potential “niche” for such cells. In other words, they were dysfunctional cells.

Yasuda further showed that adoptive transfer of normal EPC into an induced AAN murine model resulted in improvement of glomerular filtration rate and reduction in proteinuria with a three-fold decrease in mortality. This appeared to be associated with an improvement in the density of the microvasculature and a reduction of apoptosis which were paralleled by reduced plasma levels of IL-1α and β and G-CSF and an increase in VEGF. Interestingly, a single injection of EPC led to an approximately 7 fold increase in both HSC and EPC in the diseased kidneys, representing less than 2% of the injected cells.

In addition, selective engraftment of a kidney-derived, mesenchymal cell line resulted in vasculogenesis and promoted functional recovery after acute ischemic injury of the kidney (12). This adoptive transfer of EPC and mesenchymal stem cells (MSC) serves as a useful tool for mobilizing a healing response in diseased organs, and thereby affords an effective therapy. Thus, according to certain embodiments, adoptive transfer of EPC and MSC plays an important role in repairing tissues/organs by re-establishing microvasculature.

In combination with the data produced by Yasuda, et al. there is now convincing evidence in a number of experimental models of progressive kidney disease that chronic hypoxia, associated with interstitial microvascular insufficiency and obliteration is central to the progressive scarring process (9-10). According to certain embodiments, the homing of progenitor cells is augmented by local hypoxia. Evidence that adriamycin-nephropathy is associated with a decrease in glomerular and peritubular capillary density (1) indicates that, if left untreated, the kidneys will undergo scarring and loss of function. The restoration of microvasculature density with increased plasma VEGF levels following adoptive transfer of EPC, in the study of Yasuda et al., points to the reversibility of such kidney injury.

Thus, according to certain embodiments, the only prerequisite for organ (e.g. renal) regeneration after chronic injury is an adequate microvasculature supply, in which endothelial progenitor cells have a role in maintaining. When an organ (e.g. the kidney) is injured, for instance, by adriamycin, such cells malfunction and the microvasculature deteriorates in parallel with dysfunction of endothelial precursors. The ensuing local hypoxia leads to cellular loss (e.g. nephron loss) and organ fibrosis. According to certain embodiments, to reverse this process, all that is needed is to reestablish normal EPC function or to re-supply normally functioning EPC. Such normally-functioning progenitor cells could link up with resident endothelial cells to reestablish a functioning microvasculature Improved perfusion and oxygenation, would allow for the resident tubular and glomerular cells on the margins of injured areas to undergo hyperplasia and repair within the scaffolding of surviving nephrons. No tubular or glomerular cell differentiation from progenitor cells is needed for such repair, and no new nephrons need to be created. Interestingly, endothelial progenitor cells have been shown to differentiate into microvessels in vivo and, according to further embodiments, this capacity alone should, secondarily, restore all cell types in the areas surrounding restored microvessels. Therefore, according to certain embodiments, a restored microvasculature is all that is required to initiate a fully differentiated tissue response to restore chronically-injured tissue (e.g. kidney, lung, and liver).

According to further embodiments, reversal of a variety of progressive diseases of organs (e.g. kidney diseases) does not require that stem cells differentiate into phenotypes specific to the organ in order to achieve healing and regeneration. Rather, Applicant's currently disclosed strategy shows that only microvasculature restoration need serve as the common pathway to healing.

According to certain embodiments, adoptive transfer of EPC is achieved by systemic administration and does not require direct injection into any particular organ (e.g. kidney and liver).

In one embodiment, the quantity of EPC is 2.5⁻⁵×10⁸ cells for humans. In various embodiments, the quantity of EPC may be provided every one to three days. In a particular embodiment, the quantity of EPC is provided in a single does that is administered only once. One of skill in the art will readily be able to convert these dosages to dosages that are effective in mammalian subjects.

According to further embodiments, any organ or tissue, including but not limited to, lungs, kidneys, liver, heart, connective tissue, and eyes, can be treated with EPC to restore microvascular integrity.

According to certain embodiments revascularization occurs via angiogenesis and/or vasculogenesis. Angiogenesis is a process of new blood vessel development (neovascularization) from preexisting vasculature, while vasculogenesis refers to blood vessel formation from endothelial progenitors that differentiate in situ. Until recently, angiogenesis was considered the only means of adult neovascularization and vasculogenesis was thought to be limited to embryologic development. However, the existence of circulating EPC has provided evidence that postnatal vasculogenesis also occurs in adults.

### EXAMPLES

The following examples are provided to better illustrate the claimed invention and are not to be interpreted as limiting the scope of the invention. To the extent that specific materials are mentioned, it is merely for purposes of illustration and is not intended to limit the invention. One skilled in the art may develop equivalent means or reactants without the exercise of inventive capacity and without departing from the scope of the invention.

### Example 1

### Isolation and Culture of Mesenchymal Stem Cells

The fibroblast-like cell line 4E, from the kidney of adult Tie-2/GFP mouse have been isolated and cloned previously using a technique for culturing multipotent mesenchymal cells from adult tissues (12, 20-21). 4E cells can be differentiated along multiple mesodermal lineages, including adipocytes, osteoblasts, as well as endothelial cells. Analysis of the expression of surface antigens, growth factor receptors, cytoskeletal proteins, and transcription factors can reveal a pattern that was compatible with both mouse MSCs and renal stromal progenitor cells (22). 4E cells are maintained on gelatin-coated dishes in minimum essential medium (MEM) with 10% horse serum (Gem Biotech, Woodland, Calif., USA) and consistently express the above markers between passages 10 and 25.

### Example 2

### Transplantation of Endothelial Progenitor Cells into Mice

4E cells (10⁶ cells per animal) are injected intravenously via tail veins of mice that are suffering from an ischemic organ. Mice are killed at different time points between 1 and 30 days after injection of 4E cells, blood samples are obtained and kidneys are removed for further analyses.

### Example 3

### Adoptive Transfer of EPC into Mice with AAN

Mice were transfused with EPC obtained from healthy age- and gender-matched Balb/c donors. EPC were isolated, maintained and expanded as detailed above. Mice received injections of approximately 5×10⁵ cells on day 5 after adriamycin injection, at the time when no significant proteinuria was yet detectable.

### Example 4

### Immunofluorescent and Immunohistochemical Analysis

Organs, including, but not limited to kidneys, livers, and lungs, are fixed in 4% paraformaldehyde overnight at 4° C., transferred to PBS containing 30% sucrose (overnight at 4° C.), embedded in OCT (Tissue Tek; Sakura Finetek, Torrance, Calif., USA) and stored at −80° C. until analysis. Cryosections are used for immunofluorescent and immunohistochemical analysis. The identification of engrafted transplanted EPC (e.g. 4E cells) is done. Capillary loss, production, and repair are analyzed using techniques described in Chen et al. (2008).

### Example 5

### Tissue and Plasma Cytokine Measurements

Snap-frozen decapsulated organs were lysed with RIPA buffer (1×PBS, 1% Nonidet P-40, 0.5% sodium deoxylate, 0.1% SDS, and protease inhibitor), homogenized, and incubated at 4° C. for 30 min. Homogenates subsequently were centrifuged at 1500 g at 4° C. for 15 min, and supernatants and plasma were stored at −80° C. until assays are performed. Multiplexed cytokine measurements for tissue homogenates and plasma were performed using multiplex assay kit (MCYTO-70K-13, Millipore, St Charles, Mo., USA), which allows the simultaneous quantification of the following analytes: TNF-α (Tumor Necrosis Factor a), Interleukin (IL)-1α, IL-β, IL-6, KC, and IL-10. VEGF was measured using mouse single-plex VEGF Beadmates (46-196; Millipore). All analytes were tested individually and in combination to ensure that there were no cross-reactions. All measurements were performed in duplicate. The plates were analyzed using Luminex IS100 analyzer (Luminex Inc., Austin, Tex., USA). The data were saved and evaluated as median fluorescence intensity (MFI) using appropriate curve-fitting software (Luminex 100IS software version 2.3). A five-parameter logistic method with weighting was used. Cytokine and VEGF levels were corrected for the amount of protein present using the Bio-Rad protein assay (Bio-Rad Laboratories, Hercules, Calif., USA) with IgG as standard.

### Example 6

### Cell Isolation from the Kidney

For stem cell isolation, single-cell suspension was prepared from the whole kidney. Kidneys from each experimental group were placed in 2 ml of ice-cold RPMI 1640 (Invitrogen, Carlsbad, Calif.) and minced using a sterile scalpel. Digestion of the tissue was performed in collagenase II (Invitrogen) solution (1 mg/ml of RPMI 1640) for 30 min at 37° C. in 5% CO₂. Cell suspensions are passed through a 35 μm nylon sieve. Repeated digestions are performed until microscopic evaluation showed a suspension of single cells. Finally, cells were washed in PBS-BSA 1% (w/v), counted and kept on ice in the dark.

### Example 7

### FACS Analysis

FACS analysis was performed to quantify the dynamics of EPC and HSC in this model. For this analysis, 1×10⁶ cells from the single-cell suspensions were incubated with specified primary antibodies for 1 h at 4° C. in the dark. The following antibodies were used for incubation: FITC-conjugated anti-mouse CD34, PE-conjugated anti-mouse Flk-1, PE-conjugated anti-mouse CD150, FITC-conjugated anti-mouse CD117 (c-Kit) (BD Pharmingen, San Diego, Calif.). After each incubation step, cells were washed with PBS-BSA 1% (w/v) and finally fixed in 1% PFA. Data were acquired using a FACScan cytometer equipped with a 488 nm argon laser and a 635nm red diode laser and analyzed using CellQuest software (Becton Dickinson, Franklin Lakes, NJ). The set-up of FACScan was performed using unstained cells. For quantification of EPC and HSC, the number of CD34/Flk-1 and CD150/c-Kit double-positive cells within the monocytic cell population was counted.

### Example 8

### Preparation of EPC and Cell Culture

To isolate bone marrow (BM) mononuclear cells, cells were obtained by flushing the tibias and femurs of BALB/c mice with PBS and density gradient centrifugation with Histopaque-1077 (Sigma Chemical Co., St. Louis, Mo.) is performed. BM mononuclear cells were cultured in Mouse Endothelial Progenitor Cell Culture Serum Free Media (Celprogen, San Pedro, Calif.) on dishes coated with 10 μg/ml pronectin (Sigma, St Louis, Mo.). After 3 days in culture, non-adherent cells were removed, and the medium is exchanged every 2 days. Thus prepared cells were further characterized to ensure the purity of EPC population (>95% of cells were labeled by these markers) by 1) uptake of DiI-labeled acetylated low density lipoprotein (2.4 μg /ml of Dil-Actylated-LDL, Biomedical Technologies, Inc., Stoughton, Mass.), and 2) Lectin binding (25 μg /ml of Fluorescein-U/ex Europeus Lectin, Biomeda Corp., Forster City, Calif.). Colony-forming unit assay was performed according to the previously described protocol (23). Briefly, 1×10⁵ BM mononuclear cells were plated on pronectin-coated dishes and 2 weeks later colonies (>50 cells) were counted. Cells also were stained for the expression of CD31. In some in vitro experiments, mouse embryonic EPC, previously established and characterized (24), were used.

To detect apoptotic and necrotic cells, FACS analysis using fluorescein isothiocyanate-Val-Ala-Asp (OMe)-fluoromethylketone (FITC-VAD-FMK, Calbiochem, La Jolla, Calif.) and 7-Aminoactinomycin D (7-AAD, Invitrogen) was performed. Detection of cell senescence was accomplished by staining for senescence-associated beta galactosidase (SA-β-gal).

### Example 9

### Morphologic Analyses

Organs (e.g. kidneys) were collected from mice at 3 weeks after injecting the EPC for morphologic analysis. Mid-coronal kidney sections were fixed in 4% paraformaldehyde (PFA) and embedded in paraffin. Paraffin sections (4 μm thick) were stained with hematoxylin and eosin, periodic acid-Schiff and Masson's trichrome and were examined by a pathologist blinded to the origin of individual preparations. Semiquantitative grading of injury, designed to evaluate the degree of glomerular injury (segmental sclerosis, podocyte hypertrophy and proliferation) and tubulointerstitial injury (tubular casts, debris, necrosis and interstitial fibrosis) was used. The degree of injury and fibrosis score ranging from 0 to 3 is determined as follows: 0, normal kidney; 1, mild changes; 2, moderate changes; 3, severe changes. The scores were determined in each section selected at random, and >20 fields were examined under ×100 magnification.

For immunohistochemical detection of renal vasculature, cryosections were stained with endothelial-specific antibodies—CD31 (BD Phermingen, San Diego, Calif.) and vWF (Dako, Glostrup, Denmark). TUNEL staining kit (Calbiochem FragEL™ DNA Fragmentation Detection Kit, La Jolla, Calif.) was used to detect apoptotic cells in paraffin sections, according to manufacturer's instructions.

### Example 10

### Multiplex Analysis of Cyto- and Chemokines

Profiling of cyto- and chemokines and VEGF release was accomplished using multiplex analysis of the plasma obtained from experimental animals (Luminex Inc, Austin Tex.). The following parameters were determined: interleukins (IL) IL-1α, IL-1β, IL-2, IL-4, IL-5, IL-6, IL-7, IL-9, IL-10, IL-12(p70), IL-13, IL-15, IL-17, interferon-gamma (IFNγ), interferon-gamma-inducible protein (IP-10), granulocyte-colony stimulating factor (GCSF), granulocyte-macrophage-colony stimulating factor (GM-CSF), tumor necrosis factor (TNF-α), keratinocyte chemoattractant (KC), monocyte chemoattractant protein (MCP-1), macrophage inflammatory protein (MIP-1α) and regulated on activation normal T cell expressed and secreted (RANTES) as previously described in Chen et al. (2008). For measurement of plasma vascular endothelial growth factor (VEGF), ELISA Development kit (Peprotech, Rocky Hill, N.J.) with 96-well ELISA microplates (Nunc MaxiSorp, Rochester, N.Y.) and 2,2′-azinobis-(3-ethyl-benzothiazoline-6-sulfonic acid) (Sigma, St Louis, Mo.) was utilized.

Various embodiments of the invention are described above in the Detailed Description. While these descriptions directly describe the above embodiments, it is understood that those skilled in the art may conceive modifications and/or variations to the specific embodiments shown and described herein. Any such modifications or variations that fall within the purview of this description are intended to be included therein as well. Unless specifically noted, it is the intention of the inventor that the words and phrases in the specification and claims be given the ordinary and accustomed meanings to those of ordinary skill in the applicable art(s).

While particular embodiments of the present invention have been shown and described, it will be obvious to those skilled in the art that, based upon the teachings herein, changes and modifications may be made without departing from this invention and its broader aspects and, therefore, the appended claims are to encompass within their scope all such changes and modifications as are within the true spirit and scope of this invention. Furthermore, it is to be understood that the invention is solely defined by the appended claims. It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to inventions containing only one such recitation, even when the same claim includes the introductory phrases “one or more” or “at least one” and indefinite articles such as “a” or “an” (e.g., “a” and/or “an” should typically be interpreted to mean “at least one” or “one or more”); the same holds true for the use of definite articles used to introduce claim recitations. In addition, even if a specific number of an introduced claim recitation is explicitly recited, those skilled in the art will recognize that such recitation should typically be interpreted to mean at least the recited number (e.g., the bare recitation of “two recitations,” without other modifiers, typically means at least two recitations, or two or more recitations).

Accordingly, the invention is not limited except as by the appended claims.

### REFERENCES

1. Yasuda, K., et al. (2010), American Journal of Pathology, 176(4):1685-1695.
2. Challen, G. A., et al. (2006), Journal of the American Society of Nephrology, 17, 1896-1912.
3. Asakura, A., et al. (2002), Exp Hematol, 30, 1339-1345.
4. Hishikawa, K. et al. (2005), J Cell Biol, 169, 921-928.
5. Iwatani, H. et al. (2004), Kidney Int, 65, 1604-1614.
6. Schinkel, A. H., et al. (1997), Proc Natl Acad Sci USA, 94, 4028-4033.
7. Zhou, S., et al. (2001), Nat Med, 7, 1028-1034.
8. Challen, G. A., et al. (2006), Stem Cells, 24, 3-12.
9. Fine, L. G., et al. (2008), Kidney International, 74, 867-872.
10. Nangaku, M., et al. (2008), Nephron Experimental Nephrology, 110, e1-e7.
11. Little, M. H., et al. (2009), J Am Soc Nephrol, 20, 2112-2117.
12. Chen, J., et al. (2008), Kidney International, 74, 879-889.
13. Burke et al. (1997), Arch Intern Med, 137: 385-388.
14. Guest I., et al. (2000), Immunopharmacology, 46: 103-112.
15. Couture et al. (2006), Pharmacol, Rev 58: 244-258.
16. Asahara et al., (1997), Science 275, 964-7.
17. Lyden et al., (2001), Nat. Med. 7, 1194-201.
18. Kalka et al., (2000), Proc. Natl. Acad. Sci. U.S.A. 97, 3422-7.
19. Kocher et al., (2001), Nat. Med. 7, 430-6 (2001).
20. da Silva Meirelles, et al., (2006), J Cell Sci; 119: 2204-2213.
21. Lucas, et al., (1995), Wound Repair Regen 3: 449-460.
22. Plotkin M D. et al. (2006), Am J Physiol Renal Physiol 291: F902-F912.
23. Aicher A, et al. (2003), Nature Med 9: 1370-1376.
24. Hatzopoulos et al. (1998), Development, 125: 1457-1468.

What is claimed is:

1. A method of restoring function to a chronically injured tissue or organ in a subject, comprising:
providing a quantity of endothelial progenitor cells (EPC); and
administering the quantity of EPC to the subject in an amount effective to restore the function and/or architecture of the chronically injured tissue or organ.

2. The method according to claim 1, wherein the quantity of EPC is administered to the subject by adoptive transfer.

3. The method according to claim 1, wherein the quantity of EPC is administered to the subject systemically.

4. The method according to claim 1, wherein the quantity of EPC is administered directly to the tissue or organ.

5. The method according to claim 1, wherein the tissue or organ is a lung, a kidney, a liver, a heart, connective tissue, an eye, or a combination thereof.

6. The method according to claim 1, wherein the architecture comprises the microvasculature of the tissue or organ.

7. The method according to claim 1, wherein the subject is a mammal

8. The method according to claim 1, wherein the subject is a human.

9. The method according to claim 1, wherein the quantity of EPC administered is 2.5⁻⁵×10⁸ cells for a human subject.

10. The method according to claim 1, wherein the quantity of EPC is administered once, in a single injection.

11. The method according to claim 1, wherein the EPC are selected from the group consisting of endothelial precursor cells, endothelial cell precursors, hematopoietic stem cells, mesenchymal stem cells, embryonic stem cell lines, erythropoietic stem cells, young bone marrow cells and young cardiac microvascular endothelial cells.

12. A method of regenerating a chronically injured tissue or organ in a subject, comprising:
providing a quantity of endothelial progenitor cells (EPC); and
administering the quantity of EPC to the subject in an amount effective to regenerate the chronically injured tissue or organ.

13. The method according to claim 12, wherein the quantity of EPC is administered to the subject by adoptive transfer.

14. The method according to claim 12, wherein the quantity of EPC is administered to the subject systemically.

15. The method according to claim 12, wherein the quantity of EPC is administered directly to the tissue or organ.

16. The method according to claim 12, wherein the tissue or organ is a lung, a kidney, a liver, a heart, connective tissue, an eye, or a combination thereof.

17. The method according to claim 12, wherein the architecture comprises the microvasculature of the tissue or organ.

18. The method according to claim 12, wherein the subject is a mammal

19. The method according to claim 12, wherein the subject is a human.

20. The method according to claim 12, wherein the quantity of EPC administered is 2.5⁻⁵×10⁸ cells for a human subject.

21. The method according to claim 12, wherein the quantity of EPC is administered once, in a single injection.

22. The method according to claim 12, wherein the EPC are selected from the group consisting of endothelial precursor cells, endothelial cell precursors, hematopoietic stem cells, mesenchymal stem cells, embryonic stem cell lines, erythropoietic stem cells, young bone marrow cells and young cardiac microvascular endothelial cells.

23. A composition for restoring function to a chronically injured tissue or organ, comprising:
a quantity of endothelial progenitor cells (EPC) in an amount effective to restore function to the tissue or organ; and
a pharmaceutically acceptable carrier.

24. The composition of claim 23, wherein the EPC are selected from the group consisting of endothelial precursor cells, endothelial cell precursors, hematopoietic stem cells, mesenchymal stem cells, embryonic stem cell lines, erythropoietic stem cells, young bone marrow cells and young cardiac microvascular endothelial cells.

25. A composition for regenerating a chronically injured tissue or organ, comprising:
a quantity of endothelial progenitor cells (EPC) in an amount effective to regenerate the tissue or organ; and
a pharmaceutically acceptable carrier.

26. The composition of claim 25, wherein the EPC are selected from the group consisting of endothelial precursor cells, endothelial cell precursors, hematopoietic stem cells, mesenchymal stem cells, embryonic stem cell lines, erythropoietic stem cells, young bone marrow cells and young cardiac microvascular endothelial cells.

27. A composition for the regeneration of the microvasculature of a chronically injured tissue or organ, comprising:
a quantity of endothelial progenitor cells (EPC) in an amount effective to regenerate the microvasculature of the tissue or organ; and
a pharmaceutically acceptable carrier.

28. The composition of claim 27, wherein the EPC are selected from the group consisting of endothelial precursor cells, endothelial cell precursors, hematopoietic stem cells, mesenchymal stem cells, embryonic stem cell lines, erythropoietic stem cells, young bone marrow cells and young cardiac microvascular endothelial cells.
